(19) United States
(12) Patent Application Publication
(10) Pub. No.: US 2024/0366955 A1
(43) Pub. Date: Nov. 7, 2024

(54) MODULE AND DEVICE FOR TREATING OSTEOARTHRITIS USING EMITTING ELECTROMAGNETIC WAVES

(71) Applicant: REMEDEE LABS, Montbonnot-Saint-Martin (FR)

(72) Inventors: David CROUZIER, Meylan (FR); Michael FOERSTER, Corenc (FR)

(21) Appl. No.: 18/644,602

(22) Filed: Apr. 24, 2024

(30) Foreign Application Priority Data
May 3, 2023 (EP) 23315152.1

(51) Int. Cl. A61N 1/40; A61N 1/36

(52) U.S. Cl. CPC A61N 1/40; A61N 1/36021; A61N 1/3604

(57) ABSTRACT

The present invention concerns a method for treating osteoarthritis and/or one of its symptoms in a human or animal subject, which comprises the application of a portable device for transmitting electromagnetic waves to said human or animal subject, wherein said portable device is capable, when it is affixed at a surface, of transmitting waves having a power flux density of at least 0.5 mW/cm² of surface and a frequency value of between 3 and 120 gigaHertz (GHz), the device being further capable of simultaneously exposing at least 2.5 cm² of the surface to the waves.

The present invention concerns a method for treating osteoarthritis and/or one of its symptoms in a human or animal subject, which comprises the application of a portable device for transmitting electromagnetic waves to said human or animal subject, wherein said portable device is capable, when it is affixed at a surface such as skin, of transmitting waves having a power flux density of at least 0.5 mW/cm² of surface and a frequency value of between 3 and 120 gigaHertz (GHz), the device being further capable of simultaneously exposing at least 2.5 cm² of the surface to the waves.

Osteoarthritis affects 10 million people in France, i.e. 17% of the population. It is a source of pain and/or discomfort on a daily basis for nearly 7 million people. It is the second cause of disability and consultation after cardiovascular diseases in France with a total cost of more than 3.5 Billion euros. Osteoarthritis can affect all the joints of the body, but it is mainly found at the level of the rachis. It is then often silent and not disabling. Osteoarthritis of the hand, and more particularly digital osteoarthritis, is the second most frequent localization. This type of osteoarthritis is particularly disabling and is responsible for irreversible deformations of the fingers. It tends to evolve in the form of arthrosic attacks.

The osteoarthritis of the knee or gonarthrosis and the osteoarthritis of the hips or coxarthrosis are particularly invalidating because they touch the joints.

For the moment, there is no curative treatment for osteoarthritis: no medication has shown any effect on the progression of the disease. The main clinical sign of osteoarthritis is pain and the treatment is therefore based on pain treatment and on the improvement of functional handicap. The proposed treatments are based on oral analgesic treatments.

The chronic use of these treatments increases the iatrogenic risk, which is particularly important in elderly subjects, who constitute a large proportion of osteoarthritis patients.

At present, patients report insufficient relief. Therefore, there is a need for an efficient therapy of osteoarthritis. Especially, there is a need for an efficient treatment of osteoarthritis related pain management.

The inventors have now surprisingly found out that pain related to osteoarthritis can be relieved and/or decreased using electromagnetic radiation operating in the millimeter band. As shown in the examples, the pain felt by the patients is decreased, thus improving their quality of life and reducing their consumption of analgesics. Said medical device that is illustrated in the examples is easy to use and is able to efficiently reduce pain.

Therefore, an object of the invention is to efficiently treat osteoarthritis by means of electromagnetic waves, and to make the devices provided for this purpose accessible and easily usable by patients.

To this end, the invention provides a method for treating osteoarthritis and/or one of its symptoms in a human or animal subject, which comprises the application of a portable device for transmitting electromagnetic waves to said human or animal subject, wherein said portable device is capable, when it is affixed at a surface such as skin, of transmitting waves having a power flux density of at least 0.5 mW/cm² of surface and a frequency value of between 3 and 120 gigaHertz (GHz), the device being further capable of simultaneously exposing at least 2.5 cm² of the surface to the waves. By “application”, it is meant affixing said portable device to a skin surface of said human or animal subject.

Another object of the invention is to efficiently treat inflammatory rheumatisms or spondylarthritis by means of electromagnetic waves, and to make the devices provided for this purpose accessible and easily usable by patients.

The “human or animal subject”, also called “patient”, is a subject who is preferably a mammal. Preferably the subject or patient is human. Preferably, the subject or patient is afflicted by osteoarthritis.

As used herein, the terms “treat” or “treatment” means to reverse, relieve or inhibit the progress of the disorder or condition to which said term applies, or one or more symptoms of said disorder or condition.

The term “osteoarthritis” includes peripheral osteoarthritis and osteoarthritis of the spine. Preferably, the osteoarthritis is peripheral osteoarthritis. Indeed osteoarthritis of the spine is often asymptomatic and not painful. Typically, peripheral osteoarthritis may be osteoarthritis of the fingers, osteoarthritis of the lower limbs, an inflammatory rheumatism or spondylarthritis.

The term “inflammatory rheumatisms” is used for rheumatic disorders. Especially, the inflammatory rheumatisms are chronic inflammatory rheumatisms. They include many different forms of osteoarthritis that lead to painful, swollen joints. It can also affect organs, such as lungs, heart, eyes, tendons and skin.

Chronic inflammatory rheumatism (CIR) forms a large family of chronic diseases which have the common point of causing inflammatory pain in the joints (such as hands, wrists, feet, ankles, knees, elbows, shoulders or hips) and/or of the spine. These joint pains are sometimes accompanied by joint swelling (arthritis) and joint or spinal stiffness, and fatigue. However, the mechanisms of action are different from one CIR to another.

Rheumatoid arthritis and spondylarthritis (including psoriatic arthritis) are the most common CIR. Rheumatoid arthritis affects approximately 0.5% of the French population (more than 300,000 people). The spondylarthritis group also affects around 0.5% of the French population.

Preferably, the method of the invention aims to treat at least one symptom of osteoarthritis in the human or animal subject. By “symptom of osteoarthritis”, it is meant at least one symptom chosen from (i) pain, (ii) stiffness and (iii) sleep impairments. Stiffness is notably observed in the joints. Preferably, the symptom of osteoarthritis is pain and/or sleep impairments, more preferably pain.

Preferably, the symptom of osteoarthritis is nociplastic pain. By “nociplastic pain”, it is meant central hypersensitivity dissociated from peripheral stimulation. Indeed nociplastic pain is defined as central hypersensitivity, the mechanism of which is attributable to a modification of the pain control and pain modulation systems (Allaz and Suter, Revue Medicale Suisse, 23 juin 2021, De l'activation des nocicepteurs a la douleur centrale: un changement de paradigme). The response to stimulation becomes disproportionate to the level of peripheral nociceptive stimulation. This strong increase in sensitivity is attributed in particular to the traces left by the passage of pain in the nociceptive circuits. Genetic factors, a history of prolonged pain, a traumatic personal history and stress are also part of the predispositions. The neuronal plasticity involved in central sensitization makes it possible to explain the persistence of pain and this in a way dissociated from peripheral stimulation.

Preferably, the method of the invention aims to increase the quality of life of a human or animal subject afflicted by osteoarthritis. By “quality of life”, it is meant an improvement of at least one of the symptoms of osteoarthritis, optionally combined with the lack or decrease of anxiety and/or depression and/or the improvement of physical functioning.

Preferably, the treatment of osteoarthritis according to the invention is objectively measured. To reach this purpose, questionnaires may be used, that are well-established.

Typically, the pain visual analogue scale (VAS), the functional index for hand osteoarthritis (FIHOA), the sleep VAS, the 5-level EQ-5D version (EQ5D-5L), the Western Ontario and McMaster Universities Osteoarthritis Index (WOMAC), the OSWESTRY disability index and/or Patient's Global Impression of Change (PGIC) questionnaire are used, and each of them provides a corresponding score that can be objectively compared.

The pain VAS is a unidimensional measure of pain intensity, used to record patients' pain progression, or compare pain severity between patients with similar conditions. The sleep VAS is similar for measuring sleep quality.

The FIHOA or Dreiser's index is a methodologically and clinically validated index, specifically developed for hand osteoarthritis.

The EQ5D-5L is a questionnaire that essentially consists of 2 pages: the EQ-5D descriptive system and the EQ visual analogue scale (EQ VAS). The descriptive system comprises five dimensions: mobility, self-care, usual activities, pain/discomfort and anxiety/depression.

The WOMAC is a set of standardized questionnaires to evaluate the condition of patients with osteoarthritis of the knee and/or hip.

The PGIC score reflects a patient's belief about the efficacy of a given treatment.

Typically, the treatment of osteoarthritis of the fingers is measured using pain VAS, FIHOA, sleep VAS, EQ5D-5L and/or PGIC score.

The treatment of osteoarthritis of the lower limbs may be measured using pain VAS, sleep VAS, WOMAC, EQ5D-5L and/or PGIC score.

The treatment of osteoarthritis of the spine may be measured using pain VAS, sleep VAS, the OSWESTRY disability index, EQ5D-5L and/or PGIC score.

The device of the invention is preferably portable, i.e. capable and intended to be worn by a patient on a regular basis, or even continuously, without significant effort and without logistical or practical disadvantages. In addition, the power flux density of at least 0.5 mW/cm² of surface allows the treatment to be effective and to reduce pain. Finally, the frequency is a frequency band particularly effective for the treatment by millimeter waves. Preferably, the optimal effect of a treatment by millimeter waves is obtained with a frequency around 61.25 GHz and a power flux density of approximately 13 mW/cm².

The device used in the invention is relevant, because it relies on one's own resource. Thus, there is no risk of overdose, nor any other side effects. Preferably it is a wearable device such as a wristband. Said therapeutic wristband is a “at home” treatment which can be used at convenience. The patients are thus completely autonomous and responsible for their own treatment in terms of time and frequency, an aspect that is poorly addressed amongst the current solutions offered to osteoarthritis patients (or patients afflicted with an inflammatory rheumatism or spondylarthritis).

Concerning the aptitude to expose at least 2.5 cm² of surface, the device may, for example, present a module comprising several antennas transmitting waves simultaneously, the area covered by all the antennas, and therefore by the module, representing at least 2.5 cm² exposed to waves in a homogeneous way. This makes it possible to obtain a surface continuously exposed to waves in a manner sufficient to induce the expected biological response.

Alternatively, the module may be capable of discontinuously exposing 2.5 cm² to waves, that is to say, several surface portions distributed over several different locations which, all together, represent 2.5 cm² of simultaneously irradiated surface.

It should be noted that the frequency transmitted by the different antennas is not necessarily the same. The different antennas may transmit different frequencies, being supplied by different application specific integrated circuits (ASICs). Nevertheless, the frequencies remain within the band of interest.

Advantageously, the waves have a power flux density of between 5 and 35 mW/cm², preferably of between 5 and 15 mW/cm².

Thus, it is a particularly efficient power band. On the other hand, some standards do not allow a power density above a certain threshold, so that the device may be calibrated so as not to exceed this limit, if necessary.

Preferably, the surface being human or animal skin, the device comprises a unit for detecting human or animal skin, the device being capable of signaling the presence or absence of the skin to be exposed to waves, and preferably capable of determining a distance between the skin and the device.

“Exposing to the waves” also means “irradiating by the waves”.

Thus, the device transmits waves directly to the subject's skin only if the skin is detected. If the skin is not detected, or if the distance between the device and the skin is too great, no transmission takes place. This prevents sending waves in any direction and allows saving energy. It is also possible to adapt the power or other parameters of the waves transmitted based on the estimated distance between the device and the skin.

Advantageously, the device is able to be worn at least in one of the following sites:
around a wrist;
on a leg;
on an ankle;
in the back;
on an ear;
in the palm of a hand; or
more generally any site presenting a strongly innervated area.

Thus, it is affixed at one of these sites, for example, around the wrist, like a watch, so as to be worn without particular inconvenience for the patient. The device of the invention further presents the advantage of being wearable by the subject. This avoids going into a therapeutic center.

Preferably, the device comprises a rechargeable battery. Thus, it works wirelessly. Alternatively, it may operate by wire, in order to deliver higher powers or over longer durations.

Advantageously, the device comprises a unit for determining at least one data from the surface, for example, an impedance data. Thus, this is carried out after transmitting the electromagnetic waves, by measuring the output power which is based on the adaptation of relative impedance to the skin. The device thereby obtains automatically one or more characteristics of the patient's skin. Preferably, the device comprises a processing unit making it possible to deduce any variation of the determined data. Thus, the data obtained are autonomously processed by the device which itself adapts the parameters of the waves, without any intervention by the patient who only has to activate or program the wave transmission without worrying about settings and adapting the waves to his own body.

The device used in the invention may also comprise a module for transmitting electromagnetic waves, which has a total volume of less than 4 cm³, preferably less than 3 cm³, and is suitable, when it is arranged at a surface, to transmit electromagnetic waves having a power flux density of at least 0.5 mW/cm² of surface. Thus, this module is particularly miniaturized, which allows it to be integrated into portable devices or to transport and use it easily. The minimum power flux density of 0.5 mW/cm² is that from which millimeter wave treatment is effective for treating pain.

Preferably, the administration of electromagnetic waves to the subject according to the invention is made during a period of 15 minutes to 50 minutes, preferably from 30 to 50 minutes, preferably from 35 to 45 minutes, preferably from 36 to 45 minutes, preferably from 37 to 40 minutes, preferably from 38 to 45 minutes. Preferably, said administration is performed one, twice or three times a day. Preferably, said administration is performed after wake-up, and/or within one hour before bed time, and/or before or after a muscular effort, and/or before or after a static period.

The invention also provides a method for treating osteoarthritis and/or one of its symptoms in a human or animal subject, which comprises a step of transmitting electromagnetic waves towards the subject's skin, thanks to a transmitter worn by said subject, said waves having a power flux density of at least 0.5 mW/cm² of skin and a frequency between 3 and 120 GHz, preferably between 50 and 100 GHz, preferably between 60 and 95 GHz. More preferably, said waves have a frequency of between 61 and 61.5 GHz.

Advantageously, the method comprises the following steps:
a unit detects human or animal skin, and
when the unit detects that the skin is located at three millimeters or less from the transmitter, the transmitter transmits the waves.

Another predetermined distance may be considered. The distance of 3 mm corresponds to a distance close enough to allow the skin to absorb almost all of the power of the electromagnetic radiation, even if the module is not in contact with the skin, for example, when moving the device or if the latter is not affixed by being pressed against the patient's body.

Preferably, the method includes the following steps:
determining at least one impedance data of the skin; and
depending on the or each data, adapting at least one transmission parameter.

Preferably, the wave transmission takes place at least one predetermined acupuncture point of the subject.

Wearing the device on the wrist, in particular on the acupuncture point Pericardium 6, the wrist being an area particularly rich in nerve endings, makes it possible to increase the effectiveness of the millimeter wave treatment.

Advantageously, the transmission is controlled at the transmitter or by means of a device able to communicate with the transmitter through a telecommunication network.

The method of the invention may be performed using a computer program comprising code instructions capable of controlling the implementation of the steps of the method described above when it is executed on a computer.

This computer program may be included in computer software but also in an application for a mobile terminal such as a smartphone or a “smartwatch”, otherwise called “connected watch”.

The invention also provides a method of accessing the program according to the invention for downloading onto a communication network.

Lastly, the invention provides a device comprising telecommunication means and the program according to the invention. Thus, this device may be, for example, a computer or a smartphone.

According to another aspect, the method of the invention is performed using a wave transmission module, which has a total volume of less than 4 cm³, preferably less than 3 cm³, and is capable, when it is placed on a surface, to transmit electromagnetic waves having a power surface density of at least 0.5 mW/cm² of surface. Thus, this module of small size may be integrated into an easily handled device, for example, portable, such as a smartphone or a smartwatch, or be integrated in large numbers into a more complex device generating high radiation without taking up a large space within the device. In addition, starting from 0.5 mW/cm², an effect in the treatment of pain is obtained, so that only one of these modules, of small size, can allow the therapeutic treatment of a patient or serve other applications such as stress reduction, generation of a feeling of well-being or the resolution of sleep disorders, without taking up space, and with reduced cost.

Advantageously, the waves have a power surface density value of between 5 and 35 mW/cm², preferably of between 5 and 15 mW/cm². Thus, the waves transmitted comply with certain standards limiting their power towards human skin, but the power remains sufficient for an effect to be obtained, for example, a reduction in pain or a feeling of well-being.

Preferably, the waves have a frequency value between 3 and 120 GHz, preferably between 50 and 100 GHz, preferably between 60 and 95 GHz. More preferably, said waves have a frequency of between 61 and 61.5 GHz. This is a particularly effective frequency band for the treatment of pain using millimeter waves.

Advantageously, it includes a rechargeable battery. Therefore, it operates wirelessly. Alternatively, it may operate by wire, in order to deliver higher powers or over longer periods of time.

Preferably, the module is able to simultaneously expose at least 2.5 cm² of the surface to the waves. Thus, the module is capable of continuously exposing 2.5 cm² of a surface, in particular skin, to the waves. Alternatively, the module may be capable of discontinuously exposing 2.5 cm² to waves, i.e. several surface portions distributed over several different locations which, all together, represent 2.5 cm² of simultaneously irradiated surface.

For example, the module has several antennas transmitting waves simultaneously, the skin area of a patient covered by all of the antennas, and therefore by the module, representing at least 2.5 continuous cm², irradiated in a homogeneous manner. This provides a continuous irradiated surface sufficient to induce the expected biological response. It should be noted that the frequency transmitted by the different antennas is not necessarily the same. The different antennas can transmit different frequencies, being supplied by different ASICs. Nevertheless, the frequencies remain within the band of interest.

Advantageously, the module comprises a heat sink comprising at least one of the following elements:
a flexible material;
a phase-change material;
a thermal buffer;
graphite; and
an elastomeric material.

Thus, the heat sink makes it possible to minimize the heating of the module, in particular if it is integrated into a device applied to the skin of a patient, in order to maintain it at a temperature below 43° C. This, again, makes it possible to comply with certain standards but also, more simply, to avoid excessive heating of the module or of the device in which it could be integrated.

Preferably, the surface being human or animal skin, the module comprises a skin detection unit capable of signaling the presence or absence of the skin to be exposed to the waves, and, preferably capable of determining a distance separating the skin and the module.

Thus, the module transmits waves directly towards the subject's skin only if the skin is detected. If the skin is not detected, or if the distance is too great between the module and the skin, no transmission takes place. This way, sending waves in any direction is avoided and energy is saved. The power or other parameters of the waves transmitted may also be adapted according to the estimated distance between the module and the skin.

The invention further provides a portable device for transmitting electromagnetic waves, comprising a module described above.

Thus, the device can be easily worn by a human or animal patient and send waves in a predetermined manner or on command, for a therapeutic purpose, in order to generate a feeling of well-being or for any other purpose. The device is all the easier to be worn since the transmission module is small.

Preferably, the method of the invention comprises the application of a portable device for transmitting electromagnetic waves to said human or animal subject, wherein said portable device comprises:
a control module;
a transmission module comprising an application-specific integrated circuit (ASIC) housed in a ball grid array-type housing, the circuit including a frequency oscillator and a power amplifier, the transmission module having a volume less than 3 cubic centimeters,
the transmission module controlled by the control module to, when placed at a surface, transmit waves having a power flux density between 5 and 35 mW/cm², preferably between 5 and 15 mW/cm², of the surface and a frequency value of between 61 and 61.5 gigaHertz (GHz), and simultaneously expose to the waves at least 2.5 cm² of the surface, and
the transmission module including a flexible sink configured to maintain the surface exposed to the waves at a temperature below 43° C., the flexible heat sink comprising a thermal buffer.

Preferably, the device is able to be worn at least in one of the following places:
around a wrist;
on a leg;
on an ankle;
on the back;
on an ear;
in the palm of a hand;
or more generally, any place presenting a highly innervated zone.

Thus, it is affixed at one of these sites, for example, in the manner of a watch around the wrist, so as to be worn by the patient without particular inconvenience.

An electromagnetic wave transmission method is also provided according to the invention, in which a module described above, worn by a human or animal subject, transmits electromagnetic waves having a power flux density of at least 0.5 mW/cm² of skin towards the skin of the subject.

### BRIEF DESCRIPTION OF THE DRAWINGS

The accompanying drawings, which are included to provide further understanding, illustrate disclosed embodiments and together with the description serve to explain the principles of the disclosed embodiments.

In the drawings:

FIG. 1 is a block diagram of an embodiment of the invention;

FIGS. 2 and 3, 5 and 6 are illustrations of a portable device according to a first embodiment of the invention. Said device is used in examples 1 and 2 below, and is called MD or Medical Device of the invention. The MD is able to emit millimeter waves with a frequency around 61.25 GHz and a power flux density of approximately 13 mW/cm²;

FIG. 4 shows the acupuncture point Pericardium 6;

FIGS. 7 to 15 are diagrams of the components of a wave transmission module according to a first embodiment;

FIGS. 16 and 17 are illustrations of such a module respectively without and with heat sink;

FIG. 18 is an illustration of a radiation of the module of FIGS. 14 and 15;

FIGS. 19 and 20 are illustrations of use according to the second and third embodiments of the invention;

FIGS. 21 to 29 illustrate components of a module according to other embodiments of the invention.

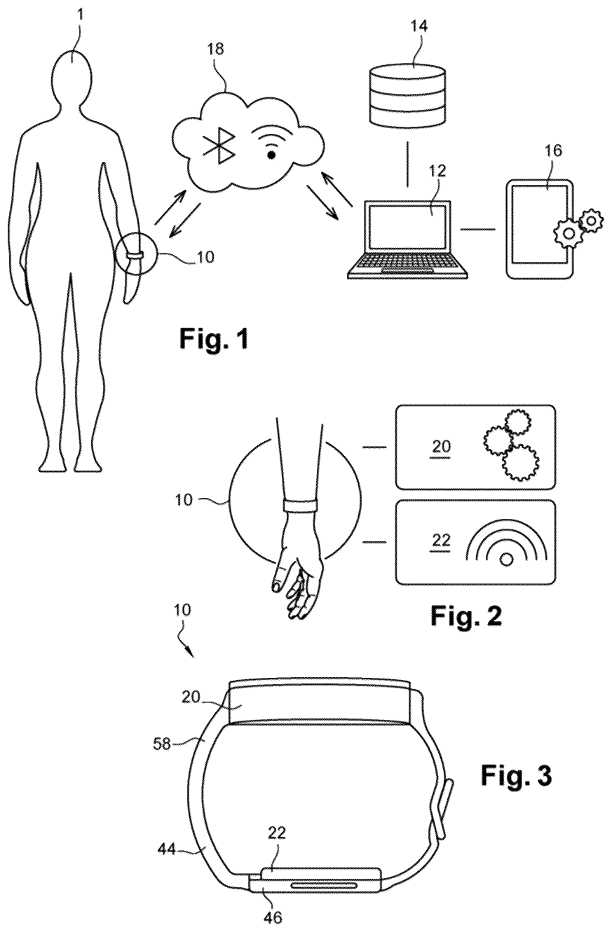

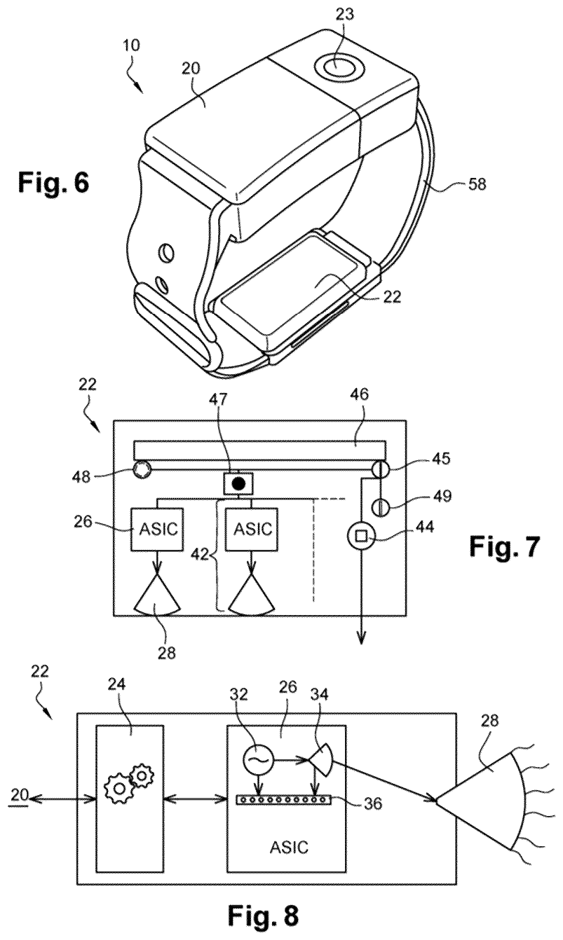

FIG. 1 illustrates the general framework of the invention. Patient 1 has chronic pain. He wears a device 10, according to a first embodiment and a first implementation of the invention, which treats the pain by transmitting electromagnetic millimeter waves to the skin of patient 1's wrist. In this case, this device 10 is in the general form of a wristwatch, and is affixed around the wrist in the same way as a watch. The device 10 comprises a control module 20 and a wave transmission module 22 illustrated schematically in FIG. 2 and in more detail in FIGS. 3, 5 and 6. The device 10 being in the general form of a watch, it may be a watch in which the modules 20 and 22 are integrated. Conversely, the functions of a watch may be integrated into device 10.

The control module 20 controls the transmission module 22. The control module 20 is activated by the patient, but it may also be programmed by the patient or another user, on the device 10 directly with the button 23 or via a terminal such as the computer 12. The button 23 is provided with light-emitting diodes which can be activated to indicate an event to the patient, for example a lack of battery or the operation of a particular program in progress. The control module 20 is present in the upper part of the device 10 while the millimeter wave transmission module 22 is located in the lower part and therefore intended to be in contact with the skin of the lower part of the wrist.

The wave transmission module 22, integrated into the device 10, will now be described in detail. It is a transmission module according to a first embodiment. This type of module, as well as its other embodiments, may be integrated into any type of device aimed at transmitting waves, and not only into the device 10 in the form of a wristwatch.

This transmission module 22, schematically illustrated in FIG. 7, presents several circuit-antenna pairs 42, a heat sink 46, a skin sensor 44, a power input 45, a digital control unit 47, a reference clock 48 and a temperature sensor 49.

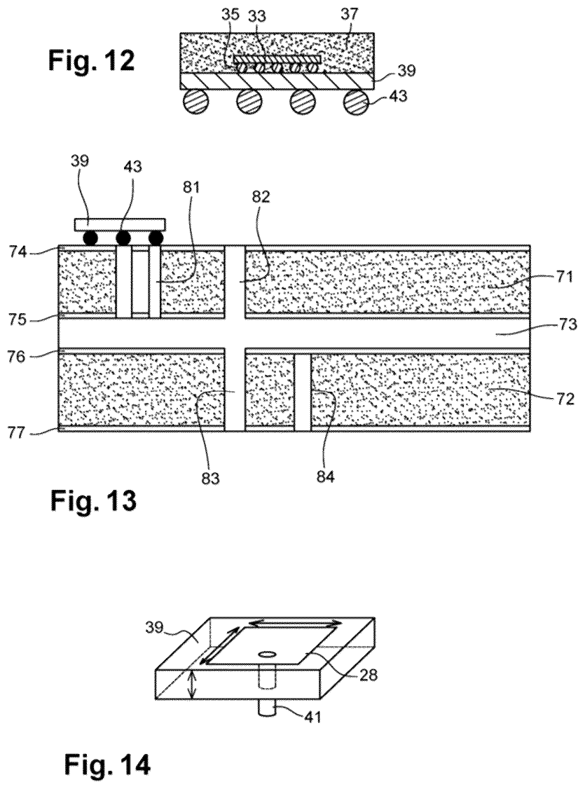

Each circuit-antenna pair 42, one of which being diagrammatically illustrated in FIG. 8, presents a control interface 24 in connection with the control module 20, an ASIC (“application-specific integrated circuit”) 26 and an antenna 28. The interface 24 may be located within the control module 20. The ASIC 26, as illustrated in FIG. 8, comprises an oscillator 32, a power amplifier 34 and a digital part 36 for setting and controlling the component. Illustrated in more detail in FIG. 9, it also includes a frequency divider 31, a communication bus 35, a “pulse-width modulation” (PWM) control unit 37 and a frequency comparator 38. The oscillator 32 allows the ASIC operating frequency to be generated. The amplifier amplifies this signal so that the desired power is available at the component output. This power is adjustable between 0 and 20 mW. It may conceivably go up to 60 mW without any difficulty. The frequency comparator and the divider make it possible to check the operating frequency. The power management circuit makes it possible to supply all of the component's functions correctly. Finally, the “PWM” control unit makes it possible to transmit the HF output signal continuously or discontinuously. The framework of the ASIC is shown in FIG. 12. This ASIC 26 is manufactured using complementary metal-oxide semiconductor (CMOS) technology, which is known to the person skilled in the art and will not therefore be described in detail. More specifically, the transistors are of the “CMOS 65 nanometer” type. Alternatively, they could have been developed in silicon-germanium (SiGe) or even in gallium arsenide (GaAs). On the other hand, technologies of the “Gunn diode” type do not allow to achieve the desired minimum size and cost. Thus, the ASIC 26 includes a silicon integrated circuit 33 housed in a ball grid array (BGA)-type housing 37, a type of housing well known to the person skilled in the art, tailor-made for the ASIC 26, the housing also comprising balls (called “bumps”) 35. As illustrated in FIG. 13, the circuit 33 is welded on two layers 71 and 72 of “HF” substrate 39 made of PTFE (Polytetrafluoroethylene) RO3003, for example manufactured by Rogers, with an arrangement known as a “flip chip”, which makes it possible to minimize the losses of electromagnetic radiation at high frequency. An alternative to RO3003 could be MT77 (for example from Isola) impregnating woven glass fibers, or even RF301 (from Taconic) or any other material offering the same technical advantages as those mentioned. The two layers 71 and 72 are separated by a layer 73 of RO4450F as well as by copper layers 74, 75, 76 and 77. In addition, vias 81, 82, 83 and 84 make the connections between the different layers of the substrate. Understandably, the types of layers and their number could be different.

The frequency oscillator 32 is placed in a cavity (not shown) within the housing 37 which allows not to disturb the generated frequency. The size of this BGA housing 37 is, in this case, 2.2×2.2×0.9 millimeters. The connection to the antennas 28 is made by means of “balls” 43. This set of components makes it possible to minimize the losses of electromagnetic waves. It is the antenna 28 which transmits electromagnetic waves to the skin of the patient 1. Needless to say, the arrangement of the ASICs, control interface and antennas within the transmission module may be different.

The terminal connection 41 between an ASIC 26 and its antenna 28 is visible in FIG. 14. Thus, a coaxial connection 41 ensures the transmission of the wave between the power amplifier 34 and the antenna 28. Antenna means generally any form of radiating element, provided that it is flat. This type of radiating element can be called a “patch”.

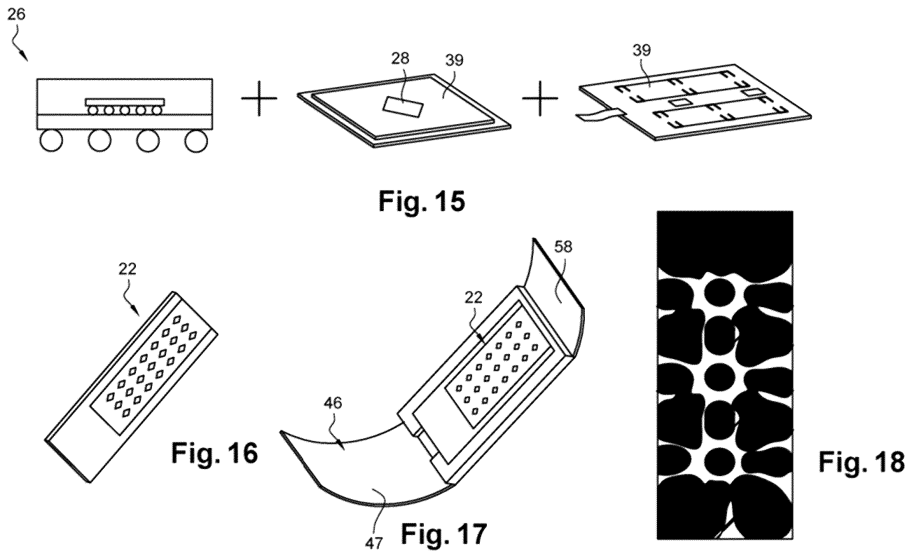

As shown in FIG. 15, the ASIC 26 and the antenna 28 are arranged on either side of the substrate 39.

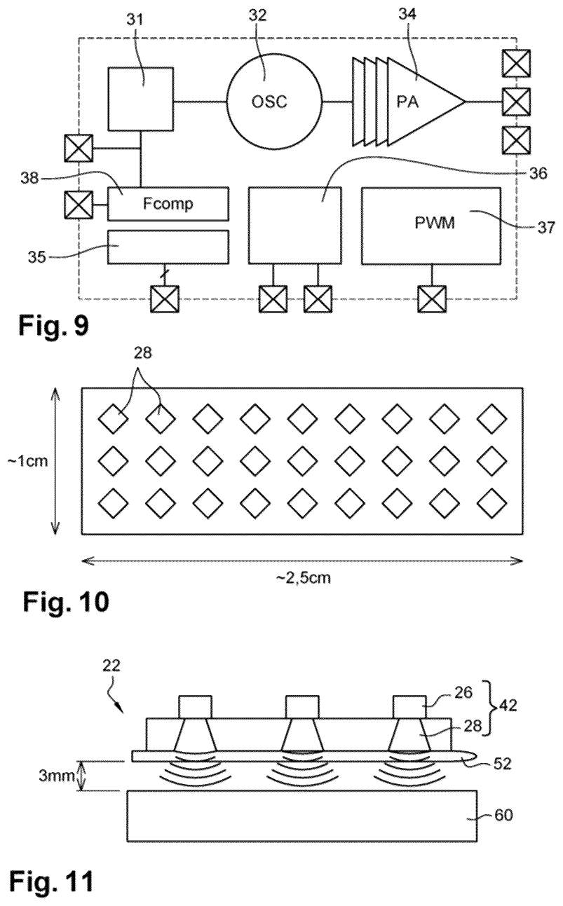

The set of antennas 28 forms an array of antennas, illustrated in FIG. 10. Rectangular-shaped here, this array of antennas, intended to be placed against the skin of patient 1 or at a short distance from it, is approximately 2.5 centimeters long by about 1 centimeter wide. It is provided, in this case, with 27 radiating elements 28 operating in near field, on the basis of three rows of nine antennas, aligned vertically and horizontally with respect to each other. These quantities and these arrangements are not limiting and others may be considered. The other elements of FIG. 7 and FIG. 9, in particular the temperature sensor 49, the skin sensor 44, the clock 48 and the power module 45 are arranged around this array of antennas, also called active area, as illustrated in FIG. 22 in a slightly different embodiment described below. The assembly formed by these elements and the active area located inside measures 37×20 mm and forms the transmission module 22, which may be integrated into a device such as a bracelet.

This arrangement allows the active area to transmit waves homogeneously over 2.5 square centimeters of skin. “Homogeneous” means that the intensity of the waves arriving on the skin must not present a deviation greater than about 30% between its maximum value at one point and its minimum value at another. FIG. 18 shows the radiation emitted by the device on the patient's skin in a normal operating mode. The black forms correspond to radiation between 5 and 15 mW/cm², the white forms to radiation below 5 mW/cm². It is observed that 75% of the surface is irradiated by waves of density between 5 and 15 mW/cm². In general, the power density can be greater than 35 mW/cm², but the device is designed so that the power range used is of the order of 5 to 35 mW/cm² in normal operation, in particular for 30 minutes of continuous wave transmission. This operating mode is indeed the most usual, as will be described below.

FIG. 11 illustrates an application of the module 22 for transmitting waves towards the skin 60 of patient 1. A distance of 3 millimeters separates the module from the skin of the patient in this case. Although the objective is to affix the device to the skin, it may happen that a slight space is created between the skin and the device. Furthermore, for more comfort and for reasons of biocompatibility, a silicone layer 52 separates the antennas from the skin, so that the skin does not have to directly support the antennas. Alternatively, it may be another material transparent to millimeter waves, such as polycarbonate. This layer 52 of silicone may measure from 1 to 2 millimeters, the design of the antennas allowing the layer 52 to have only little or no interference with the waves emitted.

Overall, this wave transmission module 22, which can be called millimeter module (the waves being said to be “millimeter” in view of their frequency) or millimeter card, measures 37 millimeters in length, 20 millimeters in width and is 3 millimeters thick in this embodiment. Therefore, the volume of the millimeter module is 2.96 cm³. As shown in FIG. 16, it is therefore less than four, and even less than three cubic centimeters, which makes it possible to insert it into light low-volume devices, such as the device 10 in the form of a wristwatch.

Having this volume and the described arrangement presenting 27 antennas, the ASICs 26 developed, coupled to the antennas 28, allow the millimeter module to transmit waves of frequency between 3 and 300 GHz, preferably between 30 and 120 GHz. The preferred frequency is 61.25 GHz+/−250 MHz. In all cases, the power flux density is at least 0.5 mW/cm², and the waves are transmitted simultaneously on a skin surface of 2.5 cm². However, a millimeter wave treatment is effective at a power density starting from 0.5 mW/cm², preferably on a surface of at least 1 cm². Therefore, the disclosed module makes it possible to carry out the treatment because it is easily integrated into any device due to its small volume.

It is understood that the ASICs, the antennas, as well as the whole of the millimeter module 22, may have different volumes, numbers and arrangements.

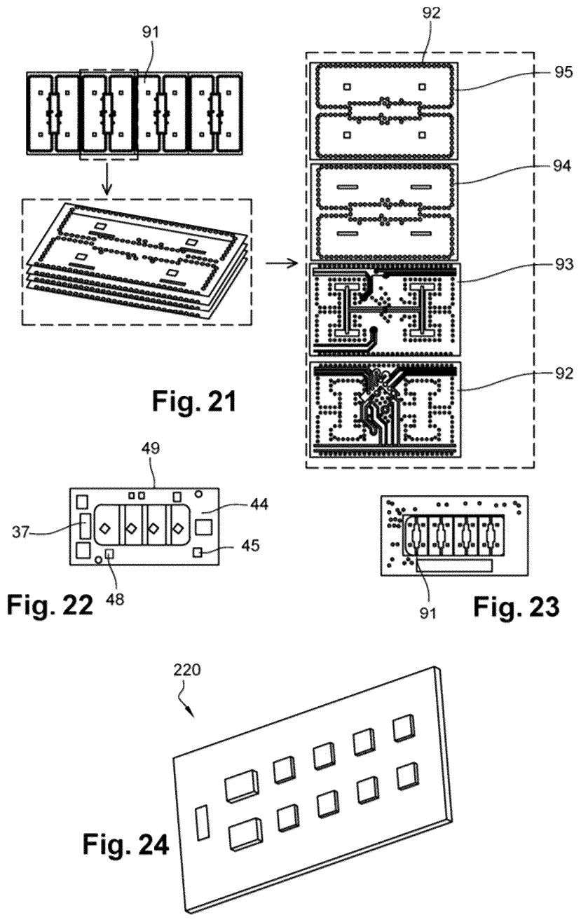

Thus, in a second embodiment, illustrated in FIGS. 21 to 23, the module's performances are identical. The difference is that an ASIC is coupled to four antennas on a surface of 10×6.25 mm. Therefore, this ASIC/antenna pair covers a skin surface of 0.625 cm², on a PCB substrate 1 mm thick. Repeated four times side by side in the millimeter module illustrated in FIGS. 21 to 23, the four ASICs are each placed in a different “BGA” housing, whose size is 2.2 mm×2.2 mm×1 mm. The module, which then includes two rows of eight antennas and 4 ASICs (4 housings), thus makes it possible to continuously irradiate 2.5 cm² of skin surface.

An antenna array 91 according to this embodiment is illustrated in FIG. 21. Array 91, said to be a “resonant cavity” array, comprises four layers. The layer 92 allows the routing of digital and power signals. The second layer 93 represents the access lines to the antennas. The third layer 94 represents the coupling lines. Lastly, the fourth layer 95 is that from which the waves will be transmitted. This antenna array is also implemented in the previous embodiment, the only difference being the number of antennas and ASICs, and, therefore, the arrangement of these elements.

Alternatively, by placing the ASIC/four antenna pairs separately at different locations on the patient's skin, this 2.5 cm² surface is irradiated, but in several distinct areas. Likewise, each of these pairs may be used independently in order to ensure greater comfort or to be integrated into applications which require a smaller surface, or a lower power.

The skin sensor 44 of the embodiments described uses a capacitive type measurement making it possible to determine that the patient's skin is positioned near the millimeter module 22. Its structure is known to the person skilled in the art and is not limited to a capacitive measurement, any miniaturizable skin sensor being admissible. Connected to the control interface 24 and/or to the control module 20, the skin sensor 44 determines the presence or absence of human or animal skin. It is also able to determine the distance between the skin and the millimeter module. At 3 millimeters or less, wave transmission is allowed. Otherwise, the control module 20 can prevent the wave transmission. The objective here is to prevent inefficient wave transmission in order, on the one hand, to control the direction of the waves transmitted, and, on the other hand, to save energy. In the first embodiment, the skin sensor 44 is located outside the module, on a side of the device 10.

The millimeter module 22 may further comprise a rechargeable battery. Preferably, the device assembly comprising the module 22, such as the device 10, has a battery supplying both the control module 20 and the wave transmission module 22. This battery can be recharged conventionally from the mains or any other way. It is, naturally, interesting that its autonomy is several hours, even several days, so that the patient's portable device aimed at treating his pain is more convenient to use.

Some of the module components may be placed outside thereof to better interact with the device comprising the module, such as the battery.

Apart from the control module 20, the millimeter module 22 and the skin sensor 44, the device 10 includes other components which will be described now.

The band 58 of FIG. 3 is flexible and aims to adapt to the shape and size of the wrist, as would a conventional watch strap.

The device 10 also includes a dissipator 46, shown in FIG. 5, which may be considered as part of the millimeter module 22. In the present case, it is located outside this module, and comprises a flexible band 47 and a thermal buffer 48, the two components being inserted within the strap of the device 10. The band 47 is associated with graphite and rubber. The rubber allows the band to be flexible and therefore adaptable to the strap. The graphite is light and has good thermal conductivity. The band 47 may be made of another elastomeric material than rubber. It may also be made of a completely different material, which needs to be flexible in order to adapt to the shape of the device. The buffer 48 includes a phase-change material. Thus, during heat release due to the operation of the device, the phase-change material absorbs part of the calories generated and allows to maintain the overall temperature. The dissipator is arranged with the device in order to maintain the temperature of the surrounding area of the body below 43° C. for a continuous operation of the device of approximately 30 minutes. This temperature of 43° C. corresponds to maximum temperature standards set by certain authorities, and that is why the device is designed to comply with these standards. It could thus be designed differently if the maximum authorized temperature were higher. The temperature is monitored by the temperature sensor of the millimeter module 22.

The device 10 further includes a unit (not shown) for determining the impedance of the skin. This unit may be part of the millimeter module 22.

The frequency of the waves transmitted by the device 10 via the module 22 may be between 3 and 300 GHz for an effective treatment. However, the frequency of the device disclosed preferably varies between 30 and 120 GHz, with a preferred frequency around 60 GHz, in particular around 61.25 GHz.

Each component's dielectric properties, such as its permittivity, conductivity and loss tangent, had to be taken into account for the design of the module 22 and the device 10. Simulations and tests outside the nominal operating range of the 65 nm CMOS type ASIC transistors were carried out, and do not call into question the lifetime of the components with regard to the implementation of the millimeter wave treatment which will be disclosed below.

The implementation of pain treatment in the patient will now be disclosed.

This treatment aims to transmit waves towards an area of the patient's skin. The transmission generally lasts 30 minutes, at the rate of one transmission to two per day. The frequency, preferably between 30 and 120 GHz, is predetermined. It may possibly vary during a transmission, as does the power flux density which generally varies between 5 and 35 mW/cm², but can be lower or higher than this range. Any other type of treatment is possible, in particular with longer and/or more frequent transmissions.

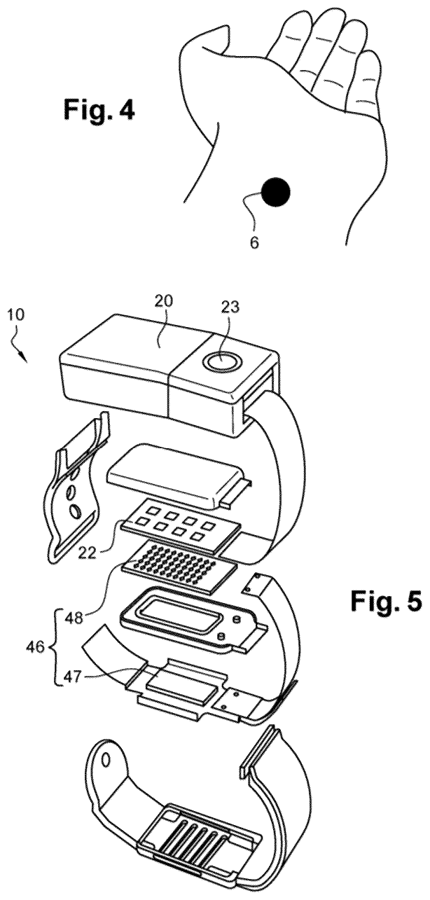

In a first embodiment, the waves are transmitted by the module 22, integrated into the device 10 in the form of a wristwatch, towards the wrist, a highly innervated area, and may be placed on the acupuncture point Pericardium 6 referenced in FIG. 4, which is a known acupuncture point. It has indeed been shown that the transmission of waves to acupuncture points was particularly effective in the treatment of pain. In addition, very good results are also achieved for particularly innervated areas. Indeed, the stimulation of the nerve endings located under the skin induces a set of physiological actions called “systemic response”, actions which in turn induce the synthesis of endogenous opioids (including the enkephalin) themselves responsible for decrease in pain. Thus, the more the wave transmission takes place in an area with a high density of nerve endings, the more the treatment is likely to be effective. Point Pericardium 6 is an acupuncture point at the same time located in an area rich in nerve endings. Therefore, a device transmitting waves towards this area is of utmost interest.

Furthermore, other potential benefits, described in the literature associated with this increase in the synthesis of opioids, are known, such as a decrease in heart rate and stress, improved sleep, or even a euphoric effect. Therefore, such benefits can be drawn from the device 10.

The frequency, the duration, and the power of the waves can be parameterized by means of the module 20 of the device 10. As illustrated in FIG. 1, it can be programmed in advance by means of a terminal, for example a computer 12, which can communicate with it by any telecommunication network, such as a Bluetooth or Wi-Fi type link 18. The computer 12 includes a database 14 on which is recorded a program 16 implementing the process or processes having a link with the invention, as well as various data allowing the implementation of the invention, in particular data entered by the patient 1 and data obtained by the device 10.

In addition, by determining the impedance of the skin using the impedance detection unit, the latter transfers to the control module 20 a characteristic data of the patient's skin. Parameters of the waves transmitted by the module 22 can then be modified automatically via the control unit 20, thanks to the program 16, or manually by the patient or another user. Thus, the device 10 adapts to the patient's skin. In other words, the electromagnetic field created is controlled by the characteristics of the skin. It can also be modified based on the distance measured between the skin and the device, via the skin detector 44. The device may include other units determining and processing other data obtained directly from the patient, which can serve to adapt the parameters of the transmitted waves such as power, frequency and duration of transmission.

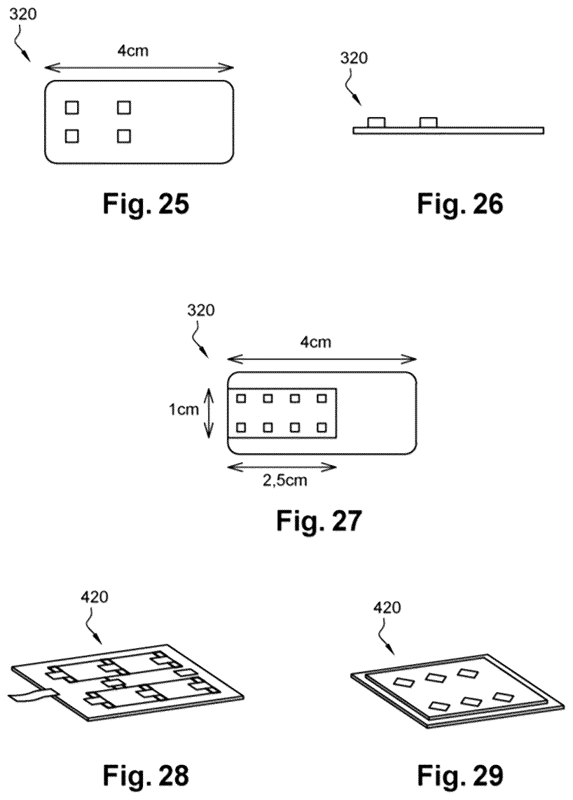

Other embodiments of the transmission module are illustrated in FIGS. 24 to 29. They differ from the previous embodiments by their number of ASICs and antennas. Thus, the module in FIG. 24 comprises 8 ASICs. In addition, one or more radiating elements may correspond to an ASIC. Thus, the module 320 comprises 4 ASICs for 8 radiating elements, at the rate of 2 radiating elements for 1 ASIC. Finally, module 420 comprises 6 ASICs and 6 radiating elements.

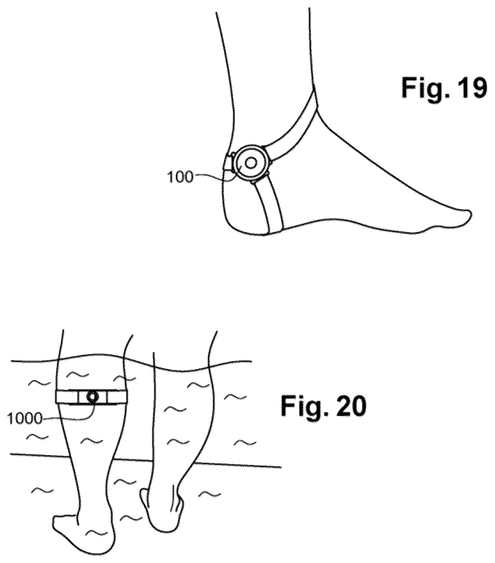

Furthermore, the transmission module may also be integrated into another device, for example intended to be worn by the patient in another part of the body. Thus, FIG. 19 illustrates a device 100 according to a second embodiment comprising the control and transmission modules placed on the ankle, while FIG. 20 illustrates such a device 1000 according to a third embodiment placed on the calf. Therefore, in these second and third embodiments, the waves are transmitted to other areas of the patient's body by means of devices which differ from the device 10 essentially to adapt to the targeted area of skin. In all cases, the miniaturization of the modules allows the device to be light and not bulky, so that it is easy to wear and not excessively burdensome.

Modifications are possible within this transmission module. For example, the structure of the antenna array may be different and present a “micro ribbon” type supply line or a coaxial probe. The antennas may be long slot antennas.

The control module may also be integrated into the electromagnetic transmission module.

Therefore, several embodiments and implementation modes were presented, which all allow the transmission of electromagnetic waves having a power surface density of at least 0.5 mW/cm² of surface, a frequency value between 3 and 120 GHz, and simultaneously on a surface of at least 2.5 cm², whether continuous or spread over several separate parts of the surface.

Aside from any pain treatment, the wave transmission module, possibly in conjunction with the control module, may be interesting for transmitting waves for other purposes, for example, to improve sleep, since it is particularly miniaturized, and therefore light. Consequently, it can be integrated into any device when it is necessary to send millimeter waves to a surface or in any direction.

Furthermore, the transmission module, or the control module, and/or the device integrating these modules, may be controlled remotely, from a terminal such as a computer, but also from a mobile terminal. For example, a mobile application comprising a pain treatment program may be saved on the mobile terminal, so that the patient programs his treatment himself, for example the power, the frequency, the duration and the time of wave transmission, or his doctor or any medical assistant programs these parameters remotely. In this case, the terminal comprises software presenting one or more interfaces allowing the user of the terminal to configure the device. The program allowing the implementation of the invention may be downloaded via a telecommunication network.

It may be added that the transmission module, as well as the device comprising it, may also be used in order to reduce the patient's stress or even bringing a feeling of well-being.

As a corollary, one can envisage the use of the transmission of electromagnetic waves within the framework of a program of improvement of a problem to be solved as perceived by the patient. The program may consist of the commitment on a series of supervised uses of the treatment with evolution of the exposure parameters (frequency, power, etc.). A discovery session, followed by a session adapted to the patient's feeling and the power of the effect perceived could be envisioned. The following sessions could also be adapted based on the measurement of said effect if sensors allow to measure it. Lastly, the treatment session could be triggered by the user through a program, or automatically if sensors allow to measure the need thereof.

According to aspects, a unit (e.g., the device 1000) is disclosed that emits millimeter waves (MMW) to a patient, which cause the central release of endogenous opioids to decrease pain and induce sleep for the patient. For example, MMWs stimulate subcutaneous nerve receptors of the patient (e.g., at the patient's wrist, or wherever skin contact occurs on the patient), sending a message to the brain, which in turn releases endorphins.

Endorphins are natural opioids and their release varies throughout the day and according to the stimulation received by the peripheral nervous system. Thus, painful (e.g. nociceptive stimuli, pregnancy and childbirth) and non-painful (e.g. temperature, massage, light) stimuli lead to an increase in endorphin levels. Endorphins are involved in pain regulation both by reducing ascending transmission of the nociceptive message and by descending inhibition from the brain to the spinal cord. At the peripheral level, endogenous opioids reduce the ascending transmission of nociceptive impulses. At the central level, endogenous opioids reduce interneural signal transmission of the nociceptive message and inhibit the release of GABA, thus resulting in abundant release of dopamine. Dopamine is a main actor in the feelings of pleasure, reward and euphoria and as such modulates the perception of pain, especially its affective and motivational aspects. Endogenous opioids also influence the balance between sympathetic and parasympathetic systems namely by inhibition of the p-adrenergic activity which results in a modulation of breathing and heartbeat. In addition, endogenous opioids also regulate the cholinergic activity, which is strongly involved in one's level of arousal. At the behavioral level, the release of endorphins triggers sleep onset.

According to certain aspects, MMW emitters may be miniaturized to fit into a wristband (e.g., the device 1000) wearable by patients for an autonomous at-home use. In an implementation, patients may perform three MMW therapy sessions of 30 minutes each, using a therapeutic wristband (e.g., the device 1000) every day for 3 months in order to improve their quality of life (e.g., less pain and better sleep). Poor sleep and pain are debilitating and strongly impact patients' quality of life. The regular use of MMW for therapy improves the patient's sleep and reduces their pain, thereby improving their quality of life. Other therapy session lengths and overall treatment durations can also be used without departing from the scope of the invention.

As an electronic medication relying on one's own resource, there's no risk of overdose, nor any other side effects. As a wearable device, the therapeutic wristband is an at-home treatment that can be used at the patient's convenience. The patients are thus completely autonomous and responsible for their own treatment in terms of time and frequency, an aspect that is poorly addressed amongst the current solutions offered to patients.

According to a specific embodiment, the method of the invention is performed as follows:

A unit detects that the device has been attached to a patient. For example, the unit may include the device 10 of FIG. 1, the device 100 of FIG. 19 or the device 1000 of FIG. 20. According to an aspect, the device may be worn by (e.g., attached to) an osteoarthritic patient, or patient afflicted with an inflammatory rheumatism or spondylarthritis.

Then, a transmitter of the device transmits electromagnetic waves to the patient during a first session. For example, the patient may wear the device on their wrist, and the device may transmit millimetric waves (MMW) through MMW emitters. Preferably, stimulating a wrist of the patient with MMW leads to central release of endogenous opioids, which decrease pain and induce sleep. The device preferably has a power flux density of at least 0.5 mW/cm² of skin and a frequency value between 3 and 120 GHz.

Then, the patient waits for a first time period, typically at least 4 hours, after the first session has ended. For example, the first session may last about 40 minutes, though other session lengths may also be employed that are greater than or less than 40 minutes. The first time period may vary depending on the patient. It may be one hour to a few hours.

Then, the transmitter of the device transmits electromagnetic waves to the patient during a second session. For example, the second session may last about 40 minutes. Other session lengths may also be employed that are greater than or less than 40 minutes, though a shorter session length may also be employed.

Then, the patient waits for a second time period, typically at least 4 hours, after the second session has ended. The second time period may vary depending on the patient. It may be one hour to a few hours.

Then, the transmitter of the device transmits electromagnetic waves to the patient during a third session. For example, the third session may last 40 minutes, though other session lengths may also be employed that are greater than or less than 40 minutes.

Preferably, at least one of the sessions may occur around or at bedtime of the patient. Preferably, the sessions may occur every day for at least 3 continuous months.

According to additional aspects, the unit (device) may track the patient's daily activities in order to determine when to deliver the treatment (e.g., a session). For example, the unit may track the patient's waking-up time, bedtime, amount of total daily activity, number of steps, etc. The unit may also average the values of the tracked daily activities. In an implementation, the unit may automatically trigger the treatment at the right time, based on how active the patient was during that day. For example, thresholds may be established for the patient based on the patient's average daily activity level. If the patient crosses any of the thresholds, then the unit may be automatically triggered to administer the treatment (e.g., begin a session). The unit may also track how often the treatment has been given in a 24-hour period, in order to prevent over-usage of the treatment. For example, the unit may be prevented from administering a treatment for a specified timeout period (e.g., four hours, or otherwise) such that it may not administer another treatment until expiry of the specified timeout. In this way, the patient may go about their day without having to actively track their own activities.

The invention also relates to a computer-implemented method for treating osteoarthritis symptoms that includes detecting, through a unit, that the unit has been attached to a patient, transmitting, through a transmitter of the unit, electromagnetic waves to the patient during a first session, waiting for a first time period, typically at least 4 hours, after the first session, transmitting, through the transmitter of the unit, electromagnetic waves to the patient during a second session, waiting for a second time period, typically at least four hours, after the second session, transmitting, through the transmitter of the unit, electromagnetic waves to the patient during a third session.

The invention also relates to a non-transitory computer-readable medium that stores instructions that, when executed by a processor, cause the processor to perform a method for treating osteoarthritis symptoms that includes detecting, through a unit, that the unit has been attached to a patient, transmitting, through a transmitter of the unit, electromagnetic waves to the patient during a first session, waiting at least four hours after the first session, transmitting, through the transmitter of the unit, electromagnetic waves to the patient during a second session, waiting at least four hours after the second session, transmitting, through the transmitter of the unit, electromagnetic waves to the patient during a third session.

Many of the above-described features and applications may be implemented as software processes that are specified as a set of instructions recorded on a computer-readable storage medium (alternatively referred to as computer-readable media, machine-readable media, or machine-readable storage media). When these instructions are executed by one or more processing unit(s) (e.g., one or more processors, cores of processors, or other processing units), they cause the processing unit(s) to perform the actions indicated in the instructions. Examples of computer-readable media include, but are not limited to, RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, ultra-density optical discs, any other optical or magnetic media, and floppy disks. In one or more embodiments, the computer-readable media does not include carrier waves and electronic signals passing wirelessly or over wired connections, or any other ephemeral signals. For example, the computer-readable media may be entirely restricted to tangible, physical objects that store information in a form that is readable by a computer. In one or more embodiments, the computer-readable media is non-transitory computer-readable media, computer-readable storage media, or non-transitory computer-readable storage media.

In one or more embodiments, a computer program product (also known as a program, software, software application, script, or code) can be written in any form of programming language, including compiled or interpreted languages, declarative or procedural languages, and it can be deployed in any form, including as a standalone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

While the above discussion primarily refers to microprocessor or multi-core processors that execute software, one or more embodiments are performed by one or more integrated circuits, such as ASICs or field programmable gate arrays (FPGAs). In one or more embodiments, such integrated circuits execute instructions that are stored on the circuit itself.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. Skilled artisans may implement the described functionality in varying ways for each particular application. Various components and blocks may be arranged differently (e.g., arranged in a different order, or partitioned in a different way), all without departing from the scope of the subject technology.

It is understood that any specific order or hierarchy of blocks in the processes disclosed is an illustration of example approaches. Based upon implementation preferences, it is understood that the specific order or hierarchy of blocks in the processes may be rearranged, or that not all illustrated blocks be performed. Any of the blocks may be performed simultaneously. In one or more embodiments, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

The present invention also relates to a method for treating osteoarthritis and/or one of its symptoms in a human or animal subject, comprising:
the application of a portable device (10; 100; 1000) for transmitting electromagnetic waves to said human or animal subject, wherein said portable device is capable, when it is affixed at a surface such as skin, of transmitting waves having a power flux density of at least 0.5 mW/cm² of surface and a frequency value of between 3 and 120 GHz, the device being further capable of simultaneously exposing at least 2.5 cm² of the surface to the waves, and
before, during and/or after said application of a portable device, a coaching step.

The present invention also relates to a method for treating osteoarthritis and/or one of its symptoms in a human or animal subject, comprising:
a step of transmitting electromagnetic waves towards the subject's skin, thanks to a transmitter worn by said subject, said waves having a power flux density of at least 0.5 mW/cm² of skin and a frequency between 3 and 120 GHz, and
before, during and/or after said step of transmitting electromagnetic waves, a coaching step.

The coaching step includes at least one of the following components:
(i) providing therapeutic education to the human or animal subject about the device or transmitter used (such as its principle of action, expected effects and/or technical aspects). Said therapeutic education aims to improve adherence, and reduce apprehension and nocebo effects of the human or animal subject. This is particularly important in osteoarthritis;
(ii) improving compliance and effectiveness, notably thanks to regular assessments of subject's usability, subject's adherence, subject's health benefits and dispensing personalized advice according to assessments, and, for example regarding food or physical activity;
(iii) discussing between subjects afflicted by osteoarthritis (i.e. peer support) or an inflammatory rheumatism or spondylarthritis, for example through physical meetings or digital forum discussions; and/or
(iv) collecting data which are to be used by the health practitioner.

The coaching step may comprise at least one discussion, by telephone, physical or through a digital platform, between the coach and the human or animal subject. The coach is preferably a trained person, preferably a nurse. Preferably, the coaching step comprises at least one discussion before said application of a portable device or said step of transmitting electromagnetic waves, and at least one discussion after said application of a portable device or said step of transmitting electromagnetic waves. The coaching step may be digitally automatically provided. Preferably, one coaching step occurs before any treatment with the device or transmitter of the invention; and at least one coaching step occurs after the first steps of treatment.

The invention is now illustrated by the examples below.

EXAMPLE 1: INNOVATIVE PAIN MANAGEMENT DEVICE USING MILLIMETRE BAND RADIATION: ELECTRONIC-PAIN KILLER—ASSESSMENT IN PATIENTS WITH PERIPHERAL OSTEOARTHRITIS—MONOCENTRIC, PROSPECTIVE, RANDOMISED IN CROSS-OVER DESIGN AND CONTROLLED TRIAL

Background: Osteoarthritis (OA) is one of the most common chronic health conditions. Painful and very disabling, it affects about 500 million people worldwide. For its management, the different guidelines recommend a combination of non-pharmacological and pharmacological treatments. However, 90% of the patients in the stop osteoarthritis I and II survey feel that they are poorly managed in terms of their osteoarthritis pain and there is a lack of effective means to do so. The inventors propose to assess an innovative medical device (MD, device of the invention, i.e. wristband) for neuromodulation of pain in patients with peripheral OA. The application of the waves transmitted by the device on the wrist, a highly innervated area, has neuromodulatory effects thanks to the synthesis and release of endorphins and the activation of the parasympathetic system.

Objectives: The inventors conducted a clinical study to evaluate if the regular use of this MD would reduce the pain felt by the patient as well as his consumption of analgesics and if it would improve quality of life (QoL).

Methods: This prospective study was performed between December 2020 and August 2022. Sixty patients with a peripheral OA and a pain score 4 on the VAS were included and randomised in one of the two cross-over group. The randomization is stratified on the most painful OA localisation (upper/lower limbs). Patients of group A followed a three-month period with their conventional treatment and after they get the device in add on for three months. Those of group B started by using for three months the MD added on conventional treatment and after they took only conventional treatment for three months. Between the 2 periods, there was a one-month wash-out with only conventional treatment. Patients performed at minimum 1 session of 40 minutes by day with the wristband and if they wished, they could add 1 or 2 other sessions. The intern memory of the MD allowed to measure this frequency of use. After 7 days of wristband use, a phone call was performed to ensure that patient had no difficulties with the wristband and that they used it correctly. A follow-up phone call was also made once a month to remind the patient to report data.

The primary outcome was the difference of pain score between the period with and without the wristband use. The primary endpoint was collected on the visual analogic scale (VAS) every day during the last two weeks of each month of follow-up (i.e. months one to three and five to seven). The other parameters, collected at the inclusion and follow-up visits are notably generic QoL with EuroQuol5D-5L questionnaire, the sleep quality (0 to 10), usability questionnaire (meCUE) and patient use of the MD. In the aim of a personalized medicine a comparison is made for each patient between the real evolution of their pain score and the decreases in the pain score that they felt having a relevant impact.

Results: All analyses were performed in intention to treat. The mean age of patients was 65.78±7.2 years old. At baseline all parameters were similar between the two groups except the number of male (group A: 5M; group B: 1M) and the body max index (group A: 24.29±4.01 kg/m²; group B: 27.80±4.7 kg/m²). The mean pain score at baseline was 6.17±1.43. For this cross-over study, there were no carryover, no sequence and no period effect. The two groups difference on the pain score was significant (p<0.05) with a VAS of 4.57±2.0 in the MD group and 5.32±1.77 in the conventional treatment group. The effect size of the MD, calculated with the Cohen's D formulae, was of 0.42. No serious adverse effect occurs.

The results can be illustrated by the following table:

| | | With MD | Without MD |
|---|---|---|---|
| Primary | Pain VAS | 4.57 ± 2.0 | 5.32 ± 1.77 |
| outcome | | | |
| Secondary | EQ5D-5L | 0.85 [0.65; 0.92] | 0.82 [0.61; 0.88] |
| outcome | (QOL) | | |
| Secondary | Sleep quality | 5.7 [4.4; 6.8] | 4.6 [3.4; 5.9] |
| outcome | VAS | | |

Conclusions: The inventors have demonstrated in this world first study the efficacy of this new medical device to reduce peripheral OA pain. Easy to use, discreet and safe, this drug-free therapy opens a new field of OA pain treatment.

This is a study: single-centre study, prospective, randomised cross-over study, comparative, open.

In the following, MD is the medical device of the invention.

### Materials & Methods

### Participants

### Eligibility Criteria

### Inclusion Criteria

Subjects meeting each of the following criteria are proposed for the study:
adult ≥50 years,
patients followed up in rheumatology consultations or at the CHUGA pain centre or in private practice for peripheral osteoarthritis (ankle, knee, hip, shoulder, elbow, digital) confirmed clinically and radiologically according to the recommendations of the American College of Rheumatology,
with a Visual Analogue Scale (VAS) pain score 4 (as an average VAS over the week prior to the inclusion visit),
patient with a stable analgesic treatment without introduction of a new therapeutic class in the last 3 months,
with a wrist size that is compatible with the MD template,
affiliated to or benefiting from a social security scheme,
who have signed a consent to participate.

### Non-Inclusion Criteria

Subjects meeting at least one of the following criteria may not be included:
with a chronic inflammatory joint disease (chronic inflammatory rheumatism, rheumatoid arthritis, psoriatic arthritis, spondyloarthritis, lupus),
patients who had received an intra-articular corticosteroid injection within the three months prior to inclusion,
with surgery planned within 8 months,
with a dermatological pathology on both wrists, such as dermatosis, oozing, hyper-sweating or an unhealed lesion,
with piercings or implanted metallic material on both wrists,
with a tattoo on both wrists,
referred to in Articles L1121-5 to L1121-8 of the Public Health Code (CSP),
in a period of exclusion from other interventional research.

### Conduct of the Research

In this single-centre, prospective study, 60 patients with peripheral osteoarthritis were randomised in a crossover design into two study arms with different treatment sequences: conventional pain treatment±daily MD sessions of one to three 40-minute sessions per day. Each treatment sequence lasts 3 months and is separated by a wash-out period of one month. The study evaluations will be carried out during the M3 and M7 visits and throughout the study with the help of a follow-up booklet given to the patient: notably evaluation of pain, quality of life and sleep and evaluation of the aptitude to use the MD. A telephone consultation will also be carried out by the investigating team one week after the start of the study and then once a month until the follow-up visit at 7 months to ensure the correct use of the MD. The patient's participation in the study is terminated at the end of this consultation.

### Objectives and Evaluation Criteria

Main objective: To compare pain with and without the use of the wristband (MD) in patients with peripheral osteoarthritis who are receiving conventional pain treatment.

Primary endpoint: Mean pain score on a visual analogue scale (VAS) obtained each month for each period of the crossover, i.e. months 1 to 3 and months 5 to 7.

The expected decrease in VAS is the clinically relevant VAS threshold of 1 point.

VAS is the most widely used and reliable scale. It is in the form of a 100 mm straight line. At one end is indicated: no pain, at the other end: maximum imaginal pain. The patient places a mark between these two extremities according to the intensity of his/her pain at a given time. The patient will rate his/her pain in average VAS at the inclusion visit (DO), at the 3-month visit and at the 7-month visit. In the home monitoring diary, the patient will assess his/her pain every day at the same time for 2 consecutive weeks per month (week 3 and 4) throughout the duration of the study.

### Objectives and Secondary Endpoints

Secondary objective 1: Comparison of the quality of life, with and without the use of the bracelet (MD), in patients with peripheral osteoarthritis who were receiving conventional pain treatment. Endpoint 1: EQ5D-5L questionnaire score at the end of each cross-over period, i.e. at 3 months (M3) and 7 months (M7).

Secondary objective 3: Comparison of the functional capacity with and without the use of the bracelet (MD) in patients with osteoarthritis of the lower limbs (hip, knee, ankle) who are receiving conventional pain treatment. Endpoint 3: WOMAC questionnaire score at the end of each of the cross-over periods, i.e. at 3 months (M3) and 7 months (M7).

Secondary objective 5: Descriptive analysis of analgesic consumption, with and without the use of the bracelet (MD), in patients with peripheral osteoarthritis receiving conventional pain treatment. Endpoint 5: Class, dose and number of times analgesics were taken at the end of each of the cross-over periods, i.e. at 3 months (M3) and 7 months (M7).

Secondary objective 8: Descriptive analysis of sleep quality with and without the use of the bracelet (MD), in patients with peripheral osteoarthritis receiving conventional pain treatment. Endpoint 8: Mean score obtained on the qualitative visual satisfaction scale recorded by the patient at the end of each of the cross-over periods, i.e. at 3 months (M3) and 7 months (M7).

Secondary Objective 9: Descriptive analysis of the use of the bracelet (MD) and its fitness for use. Judgement criterion 9: Log files of the medical device (number and duration of sessions, pause), qualitative surveys on the acceptability of the sessions (sensation, duration of sessions, . . . ), on the feeling of effectiveness of the sessions and on the aptitude for use (wristband format, material, autonomy, . . . ) at the end of each of the cross-over periods, i.e. at 3 months (M3) and at 7 months (M7).

### Sample Size

The number of subjects required was calculated for a cross-over analysis with an alpha risk (α) of 5% and a beta risk (β) of 10%.

The mean VAS expected with conventional pain management treatment is 6.1, and the expected gain for conventional treatment with the addition of the bracelet sessions is 1 VAS point, giving a mean VAS of 5.1. The standard deviation of the expected differences is 2.

With these data, a Necessary Number of Subjects (NNS) of 25 per group is calculated using the nQuery version 8 software (“t-test (ANOVA) for Difference of Means in 2×2 Crossover Design” module).

Taking into account the risk of attrition, a final NSN of 30 subjects per group was considered, i.e. a total number of 60 patients. This LOS also easily allows for the stratification envisaged for randomisation.

### Statistical Methods

### Analysis of Primary and Secondary Objectives

Main objective: Comparison of pain with and without the use of the wristband (MD) in patients with peripheral osteoarthritis receiving their conventional pain treatment.

Primary endpoint: Mean pain score on a visual analogue scale (VAS) obtained each month for each period of the crossover, i.e. months 1 to 3 and months 5 to 7.

The expected decrease in VAS is the clinically relevant VAS threshold of 1 point.

The main analysis of the main objective will be carried out by implementing a Student's t test of the difference of means from zero if the conditions for applying the test are validated, a signed ranks test otherwise. A mixed linear regression will be implemented in a second step as a first secondary analysis of the primary endpoint. The patient effect will be analysed as a random effect, the sequence and intervention effects and their interaction will be analysed as a fixed effect.

Secondary objective 1: Comparison of quality of life, with and without the use of the bracelet (MD), in patients with peripheral osteoarthritis receiving conventional pain treatment.

Secondary endpoint 1: EQ5D-5L questionnaire score at the end of each cross-over period, i.e. at 3 months (M3) and 7 months (M7).

The analysis of secondary objective 1 will be carried out by implementing a Student's t test of the difference of means from zero if the conditions for the application of the test are validated, a signed ranks test otherwise.

Secondary objective 8: Descriptive analysis of sleep quality with and without the use of the bracelet (MD), in patients suffering from peripheral osteoarthritis and benefiting from their conventional pain treatment.

Secondary endpoint 8: Mean score obtained on the qualitative visual satisfaction scale recorded by the patient at the end of each of the cross-over periods, i.e. at 3 months (M3) and 7 months (M7).

The analysis of secondary objective 8 will be carried out using the mean and standard deviation after verification of the conditions of application or by the median and the 25th and 75th percentiles according to the distribution of the data for quantitative variables and of frequencies and numbers for qualitative variables.

Sleep was assessed by a qualitative visual satisfaction scale. It is a 100 mm straight line. At one end is indicated: very good quality, at the other end: very bad quality. The patient places a mark between these two ends according to the quality of his sleep. The patient will record his/her average quality of sleep once a week every month for the duration of the study.

Secondary objective 9: Descriptive analysis of the use of the bracelet (MD) and its suitability for use.

Secondary endpoint 9: Medical device log files (number and duration of sessions, pause), qualitative surveys on session acceptability (sensation, duration of sessions, . . . ), on the feeling of effectiveness of the sessions and on the aptitude for use (bracelet format, material, autonomy . . . ) at the end of each of the cross-over periods, i.e. at 3 months (M3) and at 7 months (M7).

The main analysis of secondary objective 9 will be carried out using the mean and standard deviation after checking the conditions of application or by the median and the percentiles, 25th and 75th percentiles according to the distribution of the data for quantitative variables and of frequencies and numbers for qualitative variables.

### Results

The clinical features are in Table 1 below.

Interpretation: The group of patients in sequence 1 (without and then with the MD) has 31 compared to 29 in the group in sequence 2 (with and then without the bracelet). All variables are balanced between the groups except gender and BMI.

Of the 60 patients included, 6 were male and 5 of these were in group 1 compared to 1 in group 2.

The BMI between the two groups is somewhat unbalanced. The values of group 1 are lower than those of group 2 (4 point difference on the median).

The location of OA (stratification variable of the randomisation sequence) is relatively well balanced between the groups.

The VAS at inclusion (primary endpoint) was balanced between the groups. So was sleep quality (secondary endpoint 8).

TABLE 1
clinical features

| | Sequence 1: | Squence 2: | |
|---|---|---|---|
| | WITHOUT | WITH and | |
| | and WITH | WITHOUT | |
| | the DM | the DM | Total |
| [symbol] | N = 31 | N = 29 | N = 60 |
| Localization of [symbol] | | | |
| Lower [symbol] | 14 (46.2%) | 15 (31.7%) | 29 (48.3%) |
| Upper [symbol] | 17 (34.8%) | 14 (48.3%) | 31 (53.7%) |
| (col %) | N = 31 | N = 29 | N = 60 |
| [symbol] | | | |
| Female | 28 (83.9%) | 28 ( [symbol] ) | 54 ( [symbol] %) |
| Male | 6 (36.1%) | 1 (2.4%) | 6 ( [symbol] %) |
| (col %) | N = 31 | N = 29 | N = 60 |
| Age | | | |
| Mean (SD) | 68.82 (7.76) | 66.07 (6.67) | [symbol] (7.20) |
| Median | [symbol] | 67.00 | 66.30 |
| Q1-Q3 | [symbol] -71.00 | 62.00-72.00 | 60.00-71.23 |
| Min-Max | 31.00-78.00 | 61.00-77.00 | 61.00-78.00 |
| N | 31 | 29 | 60 |
| [symbol] | | | |
| Mean (SD) | 16.3-49 (9.34) | 161.72 (6.11) | 162.62 (7. [symbol] ) |
| Median | [symbol] | 162.00 | [symbol] |
| Q1-Q3 | 157.80- [symbol] | 137.00-167.00 | 157.00-168.00 |
| Min-Max | 145.00- [symbol] | [symbol] | 145.00-183.00 |
| N | 31 | 29 | 60 |
| Weight | | | |
| Mean (SD) | 66.00 (12.73) | 72.76 (13.34) | 68.75 (13.21) |
| Median | [symbol] | 71.00 | 68.00 |
| Q1-Q3 | [symbol] | 62.00-83.00 | 59.79-77.25 |
| Min-Max | 42 [symbol] | 47.00-98.00 | 42.00-98.00 |
| N | 31 | 29 | 60 |
| BM [symbol] | | | |
| Mean (SD) | [symbol] | 27.80 (4.70) | 23.99 (4.67) |
| Median | 23.81 | 27.89 | 26.38 |
| Q1-Q3 | 20.89-26 [symbol] | [symbol] | 22.27-29.11 |
| Min-Max | [symbol] | 16.83- [symbol] | [symbol] |
| N | 31 | 29 | 60 |
| [symbol] | | | |
| Yes | 23 ( [symbol] %) | [symbol] | 48 ( [symbol] %) |
| (col %) | N = 23 | N = 23 | N = 48 |
| [symbol] | | | |
| Yes | 27 ( [symbol] %) | [symbol] | [symbol] |
| (col %) | N = 27 | N = 25 | N = [symbol] |
| [symbol] | | | |
| [symbol] | 1 ( [symbol] %) | 1 (2.4%) | 2 (3.2%) |
| [symbol] | 16 ( [symbol] %) | 12 (41.4%) | 28 ( [symbol] .7%) |
| [symbol] | 1 (2.2%) | 2 (6.9%) | [symbol] |
| [symbol] | [symbol] | 13 (44.8%) | 22 (38.7%) |
| [symbol] | 4 (12.9%) | 1 (3.4%) | [symbol] (8.3%) |
| (col %) | N = 31 | N = 29 | N = 60 |
| [symbol] | | | |
| Grade 1 | 3 ( [symbol] %) | 2 (15.4%) | 5 (22.7%) |
| Grade 2 | 0 ( [symbol] %) | 4 (30.8%) | 4 (18.2%) |
| Grade 3 | 3 (33.3%) | [symbol] | 9 (40.7%) |
| Grade 4 | 3 (33.3%) | 1 (7.7%) | 4 (18.2%) |
| (col %) | N = [symbol] | N = 13 | N = 22 |
| Type [symbol] | | | |
| [symbol] | 1 (3.2%) | 2 (6.9%) | 3 (3.0%) |
| [symbol] | 1 (3.2%) | 1 ( [symbol] ) | 2 (3.3%) |
| [symbol] | [symbol] %) | 2 (6.9%) | 7 (11.7%) |
| [symbol] | 24 (77.4%) | 24 ( [symbol] %) | 48 ( [symbol] %) |
| (col %) | N = 31 | N = 29 | N = 60 |
| [symbol] of the diagnosis | | | |
| <3 months at | 10 (32.3%) | 15.(51.7%) | 25 (41.7%) |
| inclusion | | | |
| >3 months at | 21 (67.7%) | 14 (48.3%) | 35 (58.3%) |
| inclusion | | | |
| (col %) | N = 31 | N = 29 | N = 60 |
| Effusion | | | |
| No | 26.(90.3%) | 24 (82.5%) | 52 (86.7%) |
| Yes | 3 (9.7%) | 5 (17.2%) | 8 (13.3%) |
| (col %) | N = 31 | N = 29 | N - 60 |
| YAS at inclusion | | | |
| Mean (SD) | 6.03 (1.36) | 6.31 (1.32) | 6.17 (1.43) |
| Median | 3.80 | 6.10 | 6.03 |
| Q1-Q3 | 5.05-6.90 | 5.00-7.10 | 5.00-7.00 |
| Mean-Max | 4.00-9.00 | 4.00-9.48 | 4.00-9.40 |
| N | 31 | 29 | 60 |
| MCID at inclusion | | | |
| Mean (SD) | 3.39 (1.62) | 3.79 (1.07) | 3.58 (1.38) |
| Median | 3.35 | 4.00 | 3.60 |
| Q1-Q3 | 2.28-4.00 | 3.00-4.50 | 2.00-4.40 |
| Min-Max | 1.10-8.40 | 1.70-3.80 | 1.10-8.40 |
| N | 30 | 29 | 39 |
| Sleep quality at | | | |
| inclusion | | | |
| Mean (SD) | 4.17 (2.78) | 4.08 (2.30) | 4.13 (2.53) |
| Median | 4.50 | 3.90 | 3.95 |
| Q1-Q3 | 1.75-6.10 | 2.70-5.20 | 2.38-5.43 |
| Min-Max | 8.00-9.50 | [symbol] | 8.00-9.80 |
| N | 31 | 29 | 60 |
| [symbol] | | | |
| No | 1 (3.2%) | 1 (3.4%) | 2 (3.3%) |
| Yes | 30 (96.8%) | 28 (96.6) | 58 (96.7%) |
| (col %) | N = 31 | N = 29 | N = 60 |
| [symbol] | | | |
| [symbol] | 28 (80.6%) | 18 (62.3%) | 43 (71.7%) |
| [symbol] | 6 (19.4%) | 11 (37.9%) | 17 (28.3%) |
| (col%) | N = 31 | N = 29 | N = 60 |

[symbol]

indicates data missing or illegible when filed

### Primary Endpoint

The analysis of the primary endpoint is done in two parts:
Paired Student's t test,
Mixed model with carry-over effect taken into account (interaction between sequence order and wristband use) VAS is measured daily at weeks 3 and 4 of each month of the cross-over period (m1 to 3 and m5 to m7) in the patient's diary.

The statistical analysis is based on the average of the daily measurements for each period of the cross-over.

### Description of the VAS

TABLE 2
YAC: VAS (or EVA below) by period
of use of the MD and sequence

| | Without | With | Total |
|---|---|---|---|
| DM | N = 2520 | N = 2520 | N = 5040 |
| EVA | | | |
| Group-Sequence 1: | | | |
| WITHOUT and WITH | | | |
| the DM | | | |
| N = 2604 (51.7%) | | | |
| Mean (SD) | 5.86 (1.64) | 5.08 (2.50) | 5.48 (2.33) |
| Median | 5.80 | 5.60 | 5.70 |
| Q1-Q3 | 4.70-7.00 | 3.10-7.10 | 4.00-7.00 |
| Min-Max | 0.30-10.00 | 0.10-9.70 | 0.10-10.00 |
| N | 1302 | 1281 | 2583 |
| Group-Sequence 2: | | | |
| WITH and WITHOUT | | | |
| the DM | | | |
| N = 2436 (48.3%) | | | |
| Mean (SD) | 4.73 (2.09) | 4.03 (1.91) | 4.36 (2.03) |
| Median | 5.00 | 4.00 | 4.50 |
| Q1-Q3 | 3.00-6.40 | 2.60-5.40 | 2.80-6.00 |
| Min-Max | 0.10-9.00 | 0.10-8.80 | 0.10-9.00 |
| N | 1217 | 1208 | 2425 |
| Total | | | |
| Mean (SD) | 5.32 (2.042) | 4.57 (2.297) | 4.95 (2.204) |
| Median | 5.50 | 4.60 | 5.10 |
| Q1-Q3 | 4.00-6.70 | 2.80-6.40 | 3.30-6.60 |
| Min-Max | 0.10-10.00 | 0.10-9.70 | 0.10-10.00 |
| N | 2519 | 2489 | 5008 |

### Paired Student's Test

TABLE 3
CJP: Average pain score on a VAS (or EVA below)

| | | | | Diff. With − |
|---|---|---|---|---|
| | With | Without | | Without |
| DM | N = 60 | N = 60 | p-value | N = 60 |
| EVA | | | | |
| Mean (SD) | 4.57 (2.00) | 5.32 (1.77) | p = 0.002 | −0.74 (1.77) |
| Min-Max | 0.70-8.90 | 0.30-10.0 | (Paired | −6.43-2.40 |
| N | 60 | 60 | Sutdent t-test) | 60 |

Interpretation: The mean VAS for the period with the bracelet is 4.57 against 5.32 for the period without the bracelet. A significant difference can be observed on the mean VAS according to the use of the bracelet (MD) of the invention. The VAS is lower for the period with the MD than for the period without the MD.

The effect size (according to Cohen's D formula) is 0.42.

TABLE 4
Post-hoc: Average pain score on a VAS (or EVA below)

| | | | | Diff. With − |
|---|---|---|---|---|
| | With | Without | | Without |
| DM | N = 60 | N = 60 | p-value | N = 60 |
| EVA | | | | |
| Mean (SD) | 4.23 (2.29) | 5.33 (2.00) | p = 0 | −1.10 (2.17) |
| Min-Max | 0.29-9.14 | 0.47-10.00 | (Paired | −7.01-4.44 |
| N | 60 | 60 | Sutdent t-test) | 60 |

Interpretation: The mean VAS at the end of the period with the bracelet is 4.23 against 5.33 at the end of the period without the bracelet (i.e. difference of −1.10). A significant difference can be observed on the mean VAS depending on the use of the bracelet (MD) of the invention. The VAS is lower for the period with the MD than for the period without the MD. The effect size (using Cohen's formula D) is 0.505.

### Secondary Endpoints

Secondary endpoint 1: EQ5D-5L

The EQ5D-5L values are set between −0.5255 and 1. The higher the score, the better the quality of life.

TABLE 4
EQ5D-5L values

| | | | | Diff. With − |
|---|---|---|---|---|
| | With | Without | | Without |
| bracel [symbol] | N = 60 | N = 60 | p-value | N = 60 |
| EQ5DVal | | | | Diff. With − |
| | | | | Without |
| Median | 0.85 | 0.82 | p = 0.026 | 0.04 |
| Q1-A3 | 0.65-9.92 | 0.61-0.58 | (Wilcoxon | −0.03-0.16 |
| Min-Max | 0.03-0.98 | 0.15-0.98 | signed-rank test) | −0.41-0.56 |
| N | 60 | 60 | | 60 |

[symbol]

indicates data missing or illegible when filed

Interpretation: The median score for the “with MD” period is 0.85 compared to 0.82 for the “without MD” period. A significant difference can be observed between the groups. The EQ5D values are higher in patients with the MD than in patients without. The effect size is 0.267 (according to Cohen's d).

### Secondary Endpoint 8: Sleep Quality

Sleep quality is measured every week of each month (m1 to 3 and m5 to m7) in the patient logbook.

TABLE 10
Average Weekly Sleep Quality Score

| | With | Without | | V5 |
|---|---|---|---|---|
| DM | N = 60 | N = 60 | p-value | N = 60 |
| Average. | | | | Diff. With − |
| weekly. | | | | Without |
| sleep. | | | | |
| quality | | | | |
| Median | 5.70 | 4.60 | p = 0.004 | 0.40 |
| Q1-A3 | 4.35-6.76 | 3.39-5.86 | (Wilcoxon | −0.22-1.51 |
| Min-Max | 0.98-9.00 | 0.56-9.64 | signed-rank test) | −2.68-5.35 |
| N | 60 | 60 | | 60 |

Interpretation: The weekly sleep quality score for the “with MD” period is a median of 5.70 compared to 4.6 for the period “without the MD” (i.e. around 24% increase in sleep VAS score for the MD group of the invention). A significant difference can be observed between the periods of MD use. The “with MD” period has a better weekly sleep quality than the “without MD” period. The effect size is 0.353 (according to Cohen's d).

### Number of MD Sessions

TABLE 14
Post-hoc: Use of the MD

| | Seq. 1 | Seq. 2 | |
|---|---|---|---|
| | WITHOUTand- | WITHand- | |
| | WITH | WITHOUT | Total |
| Sequence | N = 31 | N = 29 | N = 60 |
| Number of | | | |
| sessions | | | |
| performed | | | |
| with a | | | |
| duration | | | |
| >1920 sec | | | |
| Mean (SD) | 134.03 (62.83) | 171.48 (53.52) | 152.13 (61.02) |
| Median | 147.00 | 165.00 | 161.50 |
| Q1-Q3 | 93.00-171.50 | 153.00-200.00 | 119.00-185.25 |
| Min-Max | 10.00-236.00 | 18.00-272.00 | 10.00-272.00 |
| N | 31 | 29 | 60 |
| Number of | | | |
| day in use | | | |
| Mean (SD) | 86.48 (18.71) | 88.38 (4.30) | 88.43 (13.66) |
| Median | 88.00 | 87.00 | 87.50 |
| Q1-Q3 | 84.00-90.00 | 85.00-91.00 | 84.00-90.25 |
| Min-Max | 23.00-158.00 | 83.00-98.00 | 23.00-138.00 |
| N | 31 | 29 | 60 |
| Number of | | | |
| sessions | | | |
| per day | | | |
| >1920 | | | |
| Mean (SD) | 1.53 (0.72) | 19.5 (0.62) | 1.74 (0.70) |
| Median | 1.62 | 1.92 | 1.83 |
| Q1-Q3 | 1.04-1.99 | 1.72-2.36 | 1.34-2.16 |
| Min-Max | 0.10-2.83 | 0.21-3.24 | 0.10-3.24 |
| N | 31 | 29 | 60 |

Interpretation: The 60 patients used the MD on average 1.74 times per day with a standard deviation of 0.7. In sequence 1, the average number of sessions per day was 1.55 compared to 1.95 in sequence 2.

As indicated above, in summary, the results of this clinical trial are:
1.1 pts of difference on the pain VAS scale at 3 months between treated (MD group) and untreated groups (without MD);
1.8 pts of pain reduction on the VAS scale between start and end of treatment; and
sleep improved by 24% on the sleep VAS score.

### EXAMPLE 2: COMPARISON OF THE PAIN VAS FOR THE MD OF THE INVENTION WITH EXISTING TREATMENTS

The pain VAS for MD of the invention is compared with:
existing classical chemical treatments such as mild opioids and/or paracetamol;
medical device for transcutaneous electrical neurostimulation (TENS): said device is commercialized by Sublimed, and comprises patches that need to be applied on the body before use;
a medical act performed by a doctor, i.e. viscosupplementation (injection of hyaluronic acid in a joint afflicted by osteoarthritis); and
a medical act performed at hospital, i.e. platelet-rich plasma transfusion.

The Minimal Clinically Important Difference (MCID) of pain VAS reduction is at least 1 in order to be significant.

The results show that VAS reduction is:
1 for classical chemical treatments (mild opioids, paracetamol),
between 1.7 and 2 for the MD of the invention,
2 for TENS,
between 2 and 3 for viscosupplementation, and
between 3 and 3.5 for platelet-rich plasma transfusion.

The patient's Global Impression of Change (PGIC) is around 3 for TENS, around 4 for classical chemical treatments (mild opioids, paracetamol), and around 5 for the MD of the invention. The PGIC score ranges from 1 (much worse) to 7 (much improved).

Thus, it can be concluded that:
Pain reduction measured on the VAS is superior to mild opioids and paracetamol and comparable to TENS, and
PGIC measured in osteoarthritis is superior to reference treatments such as mild opioids and TENS.

The MD of the invention is thus easy to apply (it is an easier way than TENS), does not necessitate any care professional's intervention, and is able to efficiently reduce pain.

1. Method for treating osteoarthritis and/or one of its symptoms in a human or animal subject, which comprises the application of a portable device for transmitting electromagnetic waves to said human or animal subject, wherein said portable device is capable, when it is affixed at a surface such as skin, of transmitting waves having a power flux density of at least 0.5 mW/cm² of surface and a frequency value of between 3 and 120 gigaHertz (GHz), the device being further capable of simultaneously exposing at least 2.5 cm² of the surface to the waves.

2. Method according to claim 1, wherein osteoarthritis is peripheral osteoarthritis or osteoarthritis of the spine, preferably peripheral osteoarthritis.

3. Method according to claim 1, which is for treating at least one symptom of osteoarthritis chosen from (i) pain, (ii) stiffness and (iii) sleep impairments.

4. Method according to claim 1, which is for increasing the quality of life.

5. Method according to claim 1, which is for treating at least one symptom of osteoarthritis that is pain, preferably nociplastic pain.

6. Method according to claim 1, wherein osteoarthritis is osteoarthritis of the fingers, osteoarthritis of the lower limbs, an inflammatory rheumatism or spondylarthritis.

7. Method according to claim 1, wherein the transmitting waves have a power flux density of between 5 and 35 mW/cm², preferably of between 5 and 15 mW/cm².

8. Method according to claim 1, wherein the device is worn at least in one of the following sites:
around a wrist;
on a leg;
on an ankle;
in the back;
on an ear; or
in the palm of a hand,
preferably the device is a wristband or a watch.

9. Method according to claim 1, wherein the transmission of electromagnetic waves to the human or animal subject is made during a period of 15 minutes to 50 minutes, preferably from 30 to 50 minutes, preferably from 35 to 45 minutes, preferably from 36 to 45 minutes, preferably from 37 to 40 minutes, preferably from 38 to 45 minutes, preferably said transmission is performed once, twice or three times a day.

10. Method according to claim 1, which comprises a step of transmitting electromagnetic waves towards the human or animal subject's skin, thanks to a transmitter worn by said subject, said waves having a power flux density of at least 0.5 mW/cm² of skin and a frequency between 3 and 120 GHz, preferably between 50 and 100 GHz, preferably between 60 and 95 GHz, more preferably said waves have a frequency of between 61 and 61.5 GHz.

11. Method according to claim 1, which comprises the following steps:
a unit detects human or animal skin, and
when the unit detects that the skin is located at three millimeters or less from the portable device comprising a transmitter, the transmitter transmits the waves.

12. Method according to claim 1, wherein the portable device comprises:
a control module;
a transmission module comprising an application-specific integrated circuit housed in a ball grid array-type housing, the circuit including a frequency oscillator and a power amplifier, the transmission module having a volume less than 3 cubic centimeters, the transmission module controlled by the control module to, when placed at a surface, transmit waves having a power flux density between 5 and 35 mW/cm², preferably between 5 and 15 mW/cm², of the surface and a frequency value of between 61 and 61.5 GHz, and simultaneously expose to the waves at least 2.5 cm² of the surface, and
the transmission module including a flexible sink configured to maintain the surface exposed to the waves at a temperature below 43° C., the flexible heat sink comprising a thermal buffer.

13. Method according to claim 1, which comprises the following steps:
A unit detects that the device has been attached to an osteoarthritic human or animal subject, i.e. osteoarthritic patient, or a human or animal subject afflicted with an inflammatory rheumatism or spondylarthritis;
Then, a transmitter of the portable device transmits electromagnetic waves to the patient during a first session, preferably the first session lasts about 40 minutes;
Then, the patient waits for a first time period, typically at least 4 hours, after the first session has ended;
Then, the transmitter of the device transmits electromagnetic waves to the patient during a second session, preferably the second session lasts about 40 minutes;
Then, the patient waits for a second time period, typically at least 4 hours, after the second session has ended;
Then, the transmitter of the device transmits electromagnetic waves to the patient during a third session, preferably the third session lasts about 40 minutes;
And preferably at least one of the sessions occurs around or at bedtime of the patient, and/or the sessions occur every day for at least 3 continuous months.

14. Method according to claim 1, which comprises the following steps:
the application of a portable device for transmitting electromagnetic waves to said human or animal subject, wherein said portable device is capable, when it is affixed at a surface such as skin, of transmitting waves having a power flux density of at least 0.5 mW/cm² of surface and a frequency value of between 3 and 120 GHz, the device being further capable of simultaneously exposing at least 2.5 cm² of the surface to the waves, and
before, during and/or after said application of a portable device, a coaching step.

15. Method according to claim 14, wherein the coaching step includes at least one of the following components:
(i) providing therapeutic education to the human or animal subject about the device or transmitter used;
(ii) improving compliance and effectiveness, notably thanks to regular assessments of subject's usability, subject's adherence, subject's health benefits and dispensing personalized advice according to assessments, and, for example regarding food or physical activity;
(iii) discussing between subjects afflicted by osteoarthritis (i.e. peer support) or an inflammatory rheumatism or spondylarthritis, for example through physical meetings or digital forum discussions; and/or
(iv) collecting data which are to be used by the health practitioner.

16. Method according to claim 14, wherein the coaching step comprises at least one discussion, by telephone, physical or through a digital platform, between the coach and the human or animal subject, and the coach is preferably a trained person, preferably a nurse.
